(19) United States
(12) Patent Application Publication
(10) Pub. No.: US 2017/0135555 A1
(43) Pub. Date: May 18, 2017

(54) ENDOSCOPE SYSTEM, IMAGE PROCESSING DEVICE, IMAGE PROCESSING METHOD, AND COMPUTER-READABLE RECORDING MEDIUM

(71) Applicant: OLYMPUS CORPORATION, Tokyo (JP)

(72) Inventor: Kazunori YOSHIZAKI, Tokyo (JP)

(73) Assignee: OLYMPUS CORPORATION, Tokyo (JP)

(21) Appl. No.: 15/408,621

(22) Filed: Jan. 18, 2017

Related U.S. Application Data
(63) Continuation of application No. PCT/JP2015/082313, filed on Nov. 17, 2015.

(51) Int. Cl. A61B 1/00; G02B 23/24; A61B 5/00; A61B 1/06; A61B 5/1455; A61B 5/1459; H04N 5/225; A61B 1/04

(52) U.S. Cl. CPC A61B 1/00009; H04N 5/2256; G02B 23/2484; G02B 23/2461; A61B 1/0002; A61B 1/043; A61B 1/0638; A61B 1/0653; A61B 5/14556; A61B 5/1459; A61B 5/7278; A61B 1/0005; A61B 1/00057; A61B 5/7203; A61B 1/00055; A61B 1/0646; A61B 5/7425; A61B 5/743; A61B 1/00006; H04N 2005/2255

(57) ABSTRACT

An image processing method includes: acquiring correction data for correcting first image data into second image data, the first image data being generated by an imaging device when a subject is irradiated with three rays of narrow band light having wavelength bands narrower than those of spectral sensitivity of pixels R, G, and B, and having spectrum peaks within wavelength bands of the spectral sensitivity of the pixels R, G, and B, and the second image data being deemed to be generated by the imaging device when white light is emitted; acquiring the first image data when the subject is irradiated with the three rays of narrow band light; generating color image data of the second image data using the first image data and the correction data; and calculating oxygen saturation of the subject using a pixel value R and a pixel value G included in the first image data.

### CROSS-REFERENCE TO RELATED APPLICATION

This application is a continuation of International Application No. PCT/JP2015/082313, filed on Nov. 17, 2015, the entire contents of which are incorporated herein by reference.

### BACKGROUND

1. Technical Field

The disclosure relates to an endoscope system, an image processing device, an image processing method, and a computer-readable recording medium for detecting vital information of a subject by using image data obtained by imaging the subject.

2. Related Art

In the related art, health condition of a subject is grasped by using vital information such as a heart rate, oxygen saturation, and a blood pressure as information to grasp health condition of a human in the medical field. For example, there is a known technology in which oxygen saturation of subject tissue is acquired by performing imaging while irradiating the subject tissue including blood vessels inside a body cavity with narrow band light including a wavelength band of 450 nm or less (refer to JP 2011-218135 A).

Also, there is a known technology in which oxygen saturation and a blood vessel depth are acquired by simultaneously acquiring two or more kinds of images out of plural kinds of images imaged while emitting light having wavelength bands different from one another (refer to JP 2011-200572 A).

### SUMMARY

In some embodiments, an endoscope system includes: an imaging device having a predetermined array pattern formed by using a pixel R for receiving light of a red wavelength band, a pixel G for receiving light of a green wavelength band, and a pixel B for receiving light of a blue wavelength band, and configured to perform photoelectric conversion on light received by each of the pixel R, the pixel G, and the pixel B to generate image data; a light source device configured to irradiate a subject with three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively; a recording unit configured to record correction data for correcting first image data into second image data, the first image data being generated by the imaging device when the light source device irradiates the subject with the three rays of narrow band light, and the second image data being deemed to be generated by the imaging device when white light is emitted; a color image generation unit configured to generate color image data corresponding to the second image data by using the correction data and the first image data generated by the imaging device when the light source device irradiates the subject with the three rays of narrow band light; an oxygen saturation calculation unit configured to calculate oxygen saturation of the subject by using a pixel value R of the pixel R and a pixel value G of the pixel G included in the first image data generated by the imaging device when the light source device irradiates the subject with the three rays of narrow band light; and a display device configured to display a color image corresponding to the color image data generated by the color image generation unit and the oxygen saturation calculated by the oxygen saturation calculation unit.

In some embodiments, provided is an image processing device for performing image processing on image data generated by an imaging device having a predetermined array pattern formed by using a pixel R for receiving light of a red wavelength band, a pixel G for receiving light of a green wavelength band, and a pixel B for receiving light of a blue wavelength band. The image processing device includes: an acquisition unit configured to: acquire correction data for correcting first image data into second image data, the first image data being generated by the imaging device when a subject is irradiated with three rays of narrow band light, the three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively, and the second image data being deemed to be generated by the imaging device when white light is emitted; and acquire the first image data generated by the imaging device when the subject is irradiated with the three rays of narrow band light; a color image generation unit configured to generate color image data corresponding to the second image data by using the first image data and the correction data acquired by the acquisition unit; and an oxygen saturation calculation unit configured to calculate oxygen saturation of the subject by using a pixel value R of the pixel R and a pixel value G of the pixel G included in the image data generated by the imaging device when the subject is irradiated with the three rays of narrow band light.

In some embodiments, provided is an image processing method for performing image processing on image data generated by an imaging device having a predetermined array pattern formed by using a pixel R for receiving light of a red wavelength band, a pixel G for receiving light of a green wavelength band, and a pixel B for receiving light of a blue wavelength band. The image processing device includes: acquiring correction data for correcting first image data into second image data, the first image data being generated by the imaging device when a subject is irradiated with three rays of narrow band light, the three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively, and the second image data being deemed to be generated by the imaging device when white light is emitted; acquiring the first image data generated by the imaging device when the subject is irradiated with the three rays of narrow band light; generating color image data corresponding to the second image data by using the first image data and the correction data; and calculating oxygen saturation of the subject by using a pixel value R of the pixel R and a pixel value G of the pixel G included in the first image data.

In some embodiments, provided is a non-transitory computer-readable recording medium with an executable program stored thereon for an image processing device. The image processing device is configured to perform image processing on image data generated by an imaging device having a predetermined array pattern formed by using a pixel R for receiving light of a red wavelength band, a pixel G for receiving light of a green wavelength band, and a pixel B for receiving light of a blue wavelength band. The program causes the image processing device to execute: acquiring correction data for correcting first image data into second image data, the first image data being generated by the imaging device when a subject is irradiated with three rays of narrow band light, the three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively, and the second image data being deemed to be generated by the imaging device when white light is emitted; acquiring the first image data generated by the imaging device when the subject is irradiated with the three rays of narrow band light; generating color image data corresponding to the second image data by using the first image data and the correction data; and calculating oxygen saturation of the subject by using a pixel value R of the pixel R and a pixel value G of the pixel G included in the first image data.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating a brief configuration of an endoscope system according to a first embodiment of the present invention;

FIG. 2 is a diagram schematically illustrating a structure of a color filter according to the first embodiment of the present invention;

FIG. 3 is a diagram illustrating a relation between narrow band light respectively emitted by a first light source unit, a second light source unit, and a third light source unit and respective spectral sensitivity of a pixel B, a pixel G, and a pixel R according to the first embodiment of the present invention;

FIG. 4 is a diagram schematically illustrating a calibration chart according to the first embodiment of the present invention;

FIG. 5 is a flowchart illustrating an outline of processing executed by the endoscope system according to the first embodiment of the present invention;

FIG. 6 is a diagram illustrating is an absorption property of hemoglobin in blood;

FIG. 7 is a diagram illustrating an exemplary image displayed by a display device according to the first embodiment of the present invention;

FIG. 8 is a diagram illustrating a brief configuration of an endoscope system according to a second embodiment of the present invention;

FIG. 9 is a flowchart illustrating an outline of correction data update processing executed by the endoscope system according to the second embodiment of the present invention;

FIG. 10 is a diagram illustrating a brief configuration of an endoscope system according to a third embodiment of the present invention;

FIG. 11 is a diagram illustrating a relation among narrow band light respectively emitted by a first light source unit, a second light source unit, and a third light source unit, respective spectral sensitivity of a pixel B, a pixel G, and a pixel R, and a transmission property of a notch filter according to the third embodiment of the present invention;

FIG. 12 is a flowchart illustrating an outline of processing executed by the endoscope system according to the third embodiment of the present invention;

FIG. 13A is a diagram illustrating an exemplary image displayed by a display device according to the third embodiment of the present invention;

FIG. 13B is a diagram illustrating an exemplary image displayed by a display device according to the third embodiment of the present invention;

FIG. 14 is a diagram illustrating an exemplary image according to a modified example of the first to third embodiments of the present invention;

FIG. 15 is a diagram illustrating an exemplary image according to a modified example of the first to third embodiments of the present invention;

FIG. 16 is a diagram illustrating an exemplary image according to a modified example of the first to third embodiments of the present invention; and

FIG. 17 is a diagram illustrating an exemplary image according to a modified example of the first to third embodiments of the present invention.

### DETAILED DESCRIPTION

In the following, modes for carrying out the present invention (hereinafter referred to as “embodiments”) will be described with reference to the drawings. The present invention is not limited by the embodiments described below. The same reference signs are used to designate the same elements throughout the drawings.

### First Embodiment

### Brief Configuration of Endoscope System

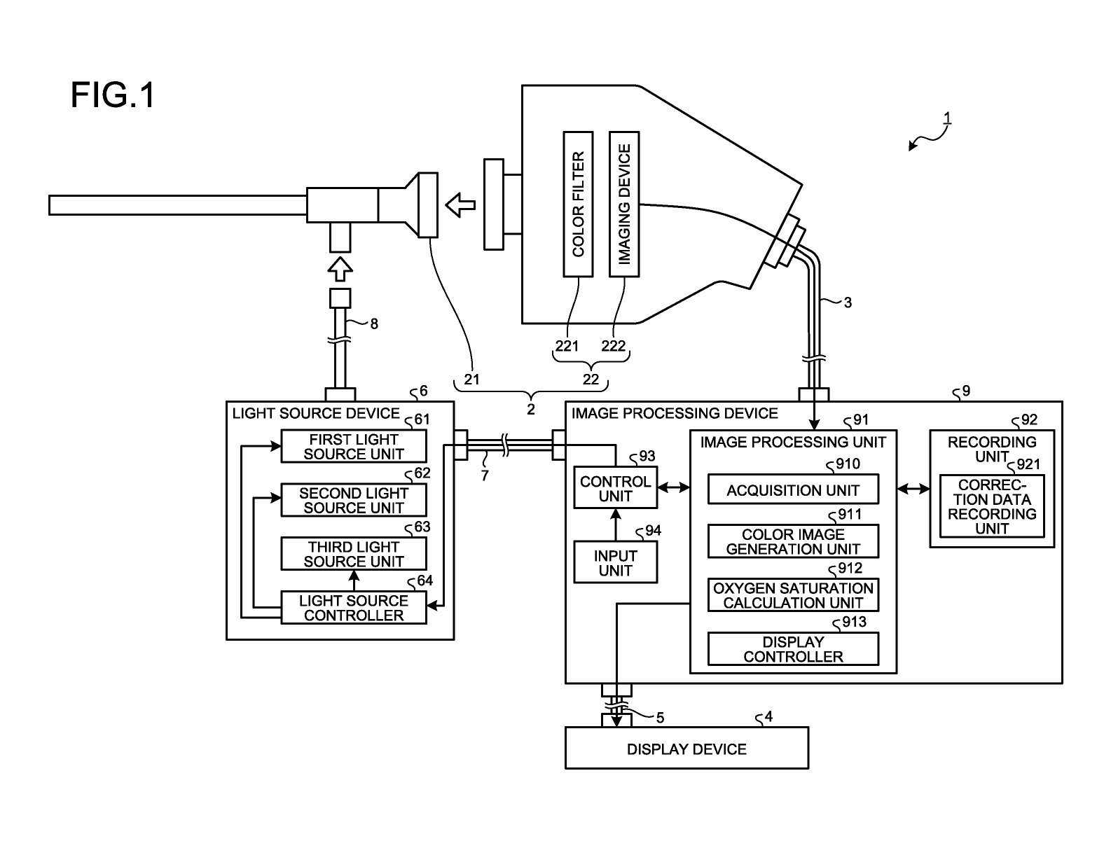

FIG. 1 is a diagram illustrating a brief configuration of an endoscope system according to a first embodiment of the present invention. An endoscope system 1 illustrated in FIG. 1 is a system used in the medical field and adapted to image and observe the inside of a subject such as a human (inside of living body). As illustrated in FIG. 1, the endoscope system 1 includes an endoscope 2, a first transmission cable 3, a display device 4, a second transmission cable 5, a light source device 6, a third transmission cable 7, a light guide 8, and an image processing device 9.

The endoscope 2 images the inside of the living body and outputs an image signal of the imaged inside of the living body. The endoscope 2 includes an inserting portion 21 and a camera head 22.

The inserting portion 21 is hard, has an elongated shape, and is configured to be inserted into the living body. An optical system formed by using one or a plurality of lenses and adapted to form a subject image is provided inside the inserting portion 21.

The camera head 22 is detachably connected to a proximal end of the inserting portion 21. The camera head 22 images a subject image formed by the optical system of the inserting portion 21 and outputs image data of this imaged subject image to the image processing device 9 under the control of the image processing device 9. The camera head 22 includes a color filter 221 and an imaging device 222.

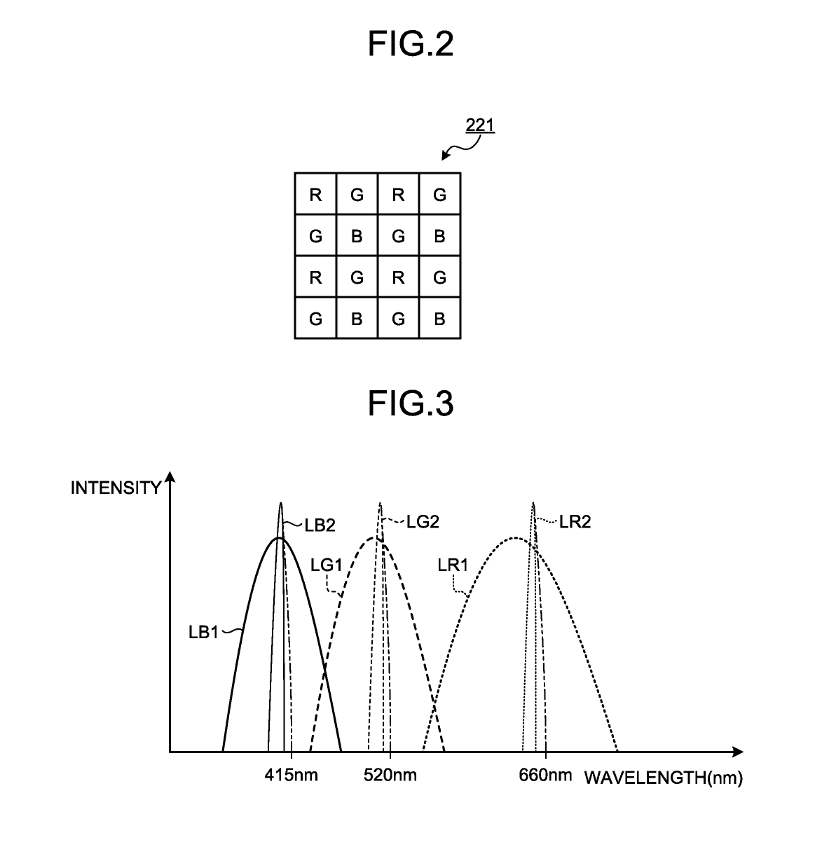

FIG. 2 is a diagram schematically illustrating a structure of the color filter 221. As illustrated in FIG. 2, the color filter 221 is formed by using a filter unit forming a predetermined array pattern (Bayer array) in which a broad band filter R adapted to pass red components, two broad band filters G adapted to pass green components, and a broad band filter B adapted to pass blue components are set as one group.

The imaging device 222 is formed by using: an image sensor such as a charge coupled device (CCD) and a complementary metal oxide semiconductor (CMOS) adapted to photoelectrically convert light received by each of a plurality of pixels arranged in a two-dimensional lattice shape and generate an image signal; and an A/D conversion circuit adapted to generate digital image data by performing A/D conversion to an analog image data (image signal) generated by the image sensor and output the same to the image processing device 9 via the first transmission cable 3. In the following, a pixel formed by arranging the broad band filter R is defined as a pixel R, a pixel formed by arranging the broad band filter G is defined as a pixel G, and a pixel formed by arranging the broad band filter B is defined as a pixel B. Instead of the A/D conversion circuit, an E/O conversion circuit may be provided which performs photoelectric conversion on an image signal to produce an optical signal, and outputs image data as the optical signal to the image processing device 9.

The first transmission cable 3 has one end detachably connected to the camera head 22 and the other end connected to the image processing device 9. The first transmission cable 3 is formed by disposing a plurality of signal lines and an optical fiber inside an outer cover that is an outermost layer.

The display device 4 displays an image corresponding to image data imaged by the endoscope 2 under the control of the image processing device 9. The display device 4 is formed by using a display panel such as a liquid crystal or an organic electro luminescence (EL).

The second transmission cable 5 has one end detachably connected to the display device 4 and the other end connected to the image processing device 9. The second transmission cable 5 transmits, to the display device 4, image data after image processing by the image processing device 9. The second transmission cable 5 is formed by using, for example, an HDMI (registered trademark), a Display Port (registered trademark), or the like.

The light source device 6 has one end connected to the light guide 8 and supplies illumination light to irradiate the inside of the living body via the light guide 8 under the control of the image processing device 9. Specifically, the light source device 6 irradiates the subject with three rays of narrow band light which are narrow band light narrower than wavelength bands of spectral sensitivity of the respective pixel R, pixel G, and pixel B, have wavelength bands different from one another, and have spectrum peaks within the wavelength bands of the spectral sensitivity of the respective pixel R, pixel G, and pixel B. The light source device 6 includes a first light source unit 61, a second light source unit 62, and a third light source unit 63, and a light source controller 64.

The first light source unit 61 emits the narrow band light having the spectrum peak in a wavelength band in which the spectral sensitivity of the pixel R is relatively high compared to the pixel G and the pixel B. Specifically, the first light source unit 61 emits the narrow band light which is narrower than the wavelength band of spectral sensitivity of the pixel R and has the spectrum peak at 660 nm. The first light source unit 61 is formed by using an LED light source, laser, and the like.

The second light source unit 62 emits the narrow band light having the spectrum peak in a wavelength band in which the spectral sensitivity of the pixel G is relatively high compared to the pixel B and the pixel R. Specifically, the second light source unit 62 emits the narrow band light which is narrower than the wavelength band of the spectral sensitivity of the pixel G and has the spectrum peak at 520 nm. The second light source unit 62 is formed by using an LED light source, laser, and the like.

The third light source unit 63 emits the narrow band light having the spectrum peak in a wavelength band in which the spectral sensitivity of the pixel B is relatively high compared to the pixel R and the pixel G. Specifically, the third light source unit 63 emits the narrow band light which is narrower than the wavelength band of the spectral sensitivity of the pixel B and has the spectrum peak at 415 nm. The third light source unit 63 is formed by using an LED, laser, and the like.

The light source controller 64 causes the first light source unit 61, second light source unit 62, and third light source unit 63 to respectively emit the light at the same time under the control of the image processing device 9. The light source controller 64 is formed by using a central processing unit (CPU) and the like.

FIG. 3 is a diagram illustrating a relation between the narrow band light respectively emitted by the first light source unit 61, second light source unit 62, and third light source unit 63 and the respective spectral sensitivity of the pixel B, pixel G, and pixel R. In FIG. 3, a horizontal axis represents a wavelength, and a vertical axis represents intensity. Furthermore, in FIG. 3, a curve LB1 represents the spectral sensitivity of the pixel B, a curve LG1 represents the spectral sensitivity of the pixel G, a curve LR1 represents the spectral sensitivity of the pixel R, a curve LB2 represents intensity of the narrow band light emitted by the third light source unit 63, a curve LG2 represents intensity of the narrow band light emitted by the second light source unit 62, and a curve LR2 represents intensity of the narrow band light emitted by the first light source unit 61.

As illustrated in FIG. 3, the first light source unit 61 emits the narrow band light having the spectrum peak at the wavelength band (660 nm) in which the spectral sensitivity of the pixel R is relatively high compared to the pixel G and the pixel B. Furthermore, the second light source unit 62 emits the narrow band light having the spectrum peak at the wavelength band (520 nm) in which the spectral sensitivity of the pixel G is relatively high compared to the pixel B and the pixel R. The third light source unit 63 emits the narrow band light having the spectrum peak at the wavelength band (415 nm) in which the spectral sensitivity of the pixel B is relatively high compared to the pixel R and the pixel G.

Referring back to FIG. 1, the explanation for the configuration of the endoscope system 1 will be continued.

The third transmission cable 7 has one end detachably connected to the light source device 6 and the other end connected to the image processing device 9. The third transmission cable 7 transmits a control signal from the image processing device 9 to the light source device 6.

The light guide 8 has one end detachably connected to the light source device 6 and the other end detachably connected to the inserting portion 21. The light guide 8 transmits the narrow band light supplied from the light source device 6 to the inserting portion 21. The light transmitted to the inserting portion 21 is emitted from a distal end of the inserting portion 21 and made to irradiate the inside of the living body. The light made to irradiate the inside of the living body is focused (collected) by the optical system inside the inserting portion 21.

The image processing device 9 is formed by using a CPU and the like, and integrally controls operation of the light source device 6, camera head 22, and display device 4. The image processing device 9 includes an image processing unit 91, a recording unit 92, a control unit 93, and an input unit 94.

The image processing unit 91 performs image processing on an image signal output from the camera head 22 via the first transmission cable 3, and outputs the image signal after the image processing to the display device 4. The image processing unit 91 includes an acquisition unit 910, a color image generation unit 911, an oxygen saturation calculation unit 912, and a display controller 913.

The acquisition unit 910 acquires image data generated by the imaging device 222 and correction data recorded by a correction data recording unit 921. Specifically, the acquisition unit 910 acquires: correction data to correct first image data generated by the imaging device 222 when the light source device 6 emits the light of the plurality of narrow bands to a subject to second image data that can be deemed to be generated by the imaging device 222 when white light is emitted; and first image data generated by the imaging device 222 when the light source device 6 emits the light of the plurality of narrow bands to the subject. The light of the plurality of narrow bands is narrower than the wavelength bands of the spectral sensitivity of the respective pixel R, pixel G, and pixel B, have wavelength bands different from one another, and have the spectrum peaks within the wavelength bands of the spectral sensitivity of the respective the pixel R, pixel G, and pixel B.

The color image generation unit 911 generates color image data corresponding to the second image data by using: the first image data generated by the imaging device 222 when the light source device 6 emits the light of the plurality of narrow bands to the subject; and the correction data recorded by the correction data recording unit 921.

The oxygen saturation calculation unit 912 calculates the oxygen saturation of the subject by using a pixel value R of the pixel R and a pixel value G of the pixel G included in the first image data generated by the imaging device 222 when the light source device 6 emits the light of the plurality of narrow bands to the subject.

The display controller 913 controls a display style of the display device 4. Specifically, the display controller 913 superimposes the oxygen saturation calculated by the oxygen saturation calculation unit 912 on a color image corresponding to the color image data generated by the color image generation unit 911, and causes the display device 4 to display the superimposed image.

The recording unit 92 records various kinds of programs executed by the image processing device 9, image data under processing, and image data. The recording unit 92 is formed by using a random access memory (RAM), a flash memory, and the like. Furthermore, the recording unit 92 includes the correction data recording unit 921.

The correction data recording unit 921 records the correction data to correct the first image data generated by the imaging device 222 when the light source device 6 emits the light of the plurality of narrow bands to the subject to the second image data that can be deemed to be generated by the imaging device 222 when white light is emitted. Details of the correction data will be described later.

The control unit 93 is formed by using a CPU and the like. The control unit 93 integrally controls the respective units of the image processing device 9. The control unit 93 controls operation of the display device 4, light source device 6, and camera head 22 in accordance with a command signal received from the input unit 94.

The input unit 94 receives input of the command signal in accordance with operation from the outside. The input unit 94 is formed by using interfaces such as a keyboard and a mouse, a switch, and the like.

Details of Correction Data

Next, the correction data recorded by the correction data recording unit 921 will be described.

In the first embodiment, since the light source device 6 emits three kinds of narrow band light, color reproducibility of image data of a subject generated by the imaging device 222 may be inferior to image data generated by the imaging device 222 when white light is emitted by a white light source in the related arts. Therefore, in the first embodiment, the correction data to achieve output deemed to be provided when white light is emitted by the white light source is calculated by a jig, a calibration portion, and the like not preliminarily illustrated, and a calculation result of this calculation is recorded in the correction data recording unit 921 as the correction data.

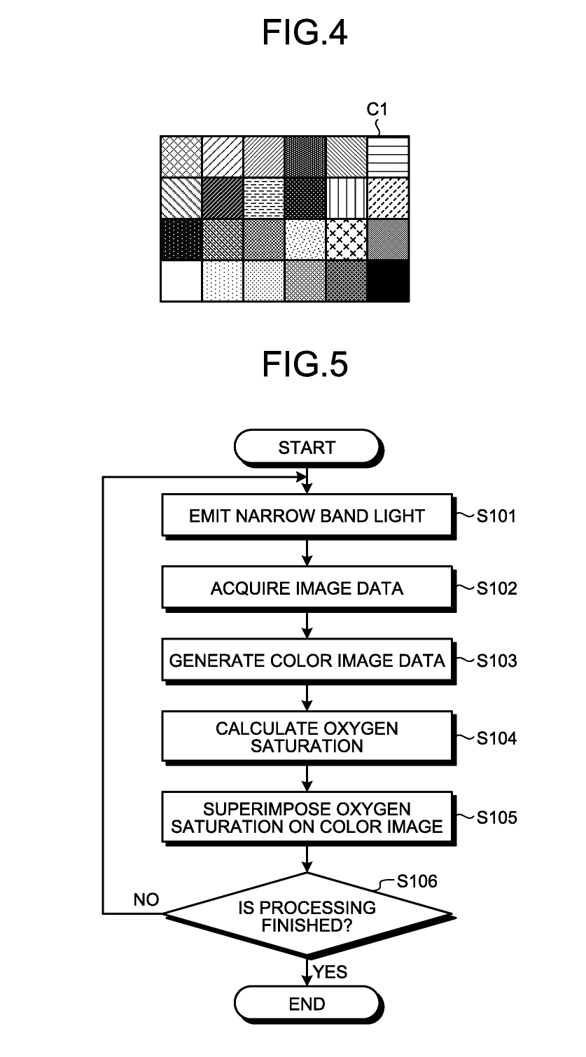

Next, a method for calculating the correction data will be described. There are various methods in order to obtain the correction data. As a method thereof, as illustrated in FIG. 4, ideal white light (uniform white light) is emitted by a white light source to a calibration chart C1 (e.g., Macbeth color checker patches and Munsell chips) that includes a plurality of color patches having a known spectrum, and the calibration chart C1 is imaged by the endoscope 2 or the imaging device 222. In this case, when sRGB data as image data imaged by the endoscope 2 or the imaging device 222 is defined as d_{sRGB}, the sRGB data can be expressed as follows.

d_{sRGB}=CR^{t}h (1)

Here, d_{sRGB} represents 3×n matrix (sRGB), C represents 3×3 matrix (XYZ→sRGB), R represents m×3 matrix (spectrum (m data)→XYZ), and h represents m×n matrix (spectrum data (number of color patches n)). R^{t} represents a transposed matrix of R.

In the case where the light source device 6 simultaneously emits the three kinds of narrow band light to the calibration chart C1 and the calibration chart C1 is imaged by the endoscope 2 or the imaging device 222, when the sRGB data as the image data imaged by the endoscope 2 or the imaging device 222 is defined as d, the sRGB data can be expressed as follows.

d=S^{t}Lh (2)

Here, S represents m×3 matrix (sensitivity of imaging device 222), and L represents m×m diagonal matrix (light source device 6). S^{t} represent a transposed matrix of S.

According to the formulas (1) and (2),

d_{sRGB}=CR^{t}[S^{t}L]⁻¹d (3)

Here, in the case of M=CR^{t} [S^{t}L]⁻¹, the following formula (4) is satisfied.

d_{sRGB}=Md (4)

Here, [S^{t}L]⁻¹ represents an inverse matrix of S^{t}L.

Thus, M is calculated by using the white light source not illustrated and the calibration chart C1, and the M is recorded in the correction data recording unit 921 as the correction data.

Operation of Endoscope System

Next, processing executed by the endoscope system 1 will be described. FIG. 5 is a flowchart illustrating an outline of the processing executed by the endoscope system 1.

As illustrated in FIG. 5, the light source device 6 first causes the first light source unit 61, second light source unit 62, and third light source unit 63 to perform light emission under the control of the image processing device 9, thereby emitting the three kinds of narrow band light at the same time (Step S101).

Subsequently, the acquisition unit 910 acquires an image signal from the camera head 22 via the first transmission cable 3 (Step S102). In this case, the acquisition unit 910 also acquires correction data from the correction data recording unit 921.

After that, the color image generation unit 911 generates a color image by using the image data acquired from the camera head 22 (Step S103). Specifically, the color image generation unit 911 generates color image data Iₒᵤₜₚᵤₜ by executing a following formula (5) using the correction data M acquired by the acquisition unit 910 from the correction data recording unit 921 and image data Iᵢₙₚᵤₜ acquired by the acquisition unit 910 from the camera head 22. Needless to say, the color image generation unit 911 generates the color image data by performing predetermined image processing, for example, image processing like demosaicing.

Iₒᵤₜₚᵤₜ=M×Iᵢₙₚᵤₜ (5)

Subsequently, the oxygen saturation calculation unit 912 calculates oxygen saturation by using a signal G (pixel value G) corresponding to the pixel G and a signal R (pixel value R) corresponding to the pixel R included in the image data (Step S104).

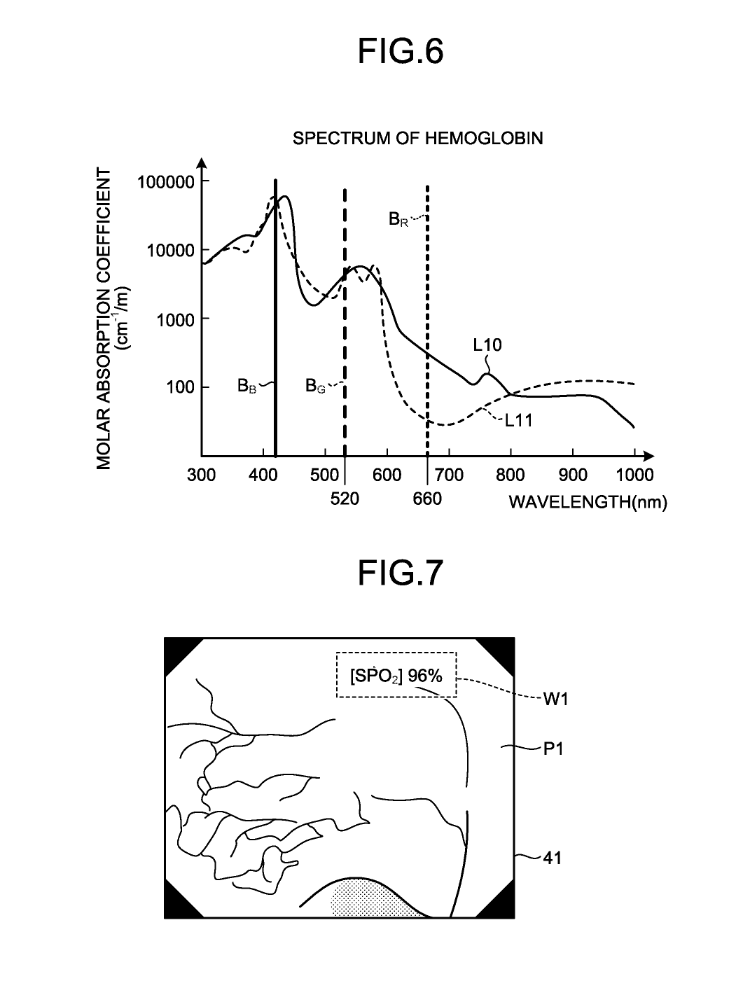

FIG. 6 is a diagram illustrating is an absorption property of hemoglobin in blood. In FIG. 6, a horizontal axis represents a wavelength (nm), and a vertical axis represents molar absorption coefficient (cm⁻¹/m). In FIG. 6, a curve L10 represents reduced hemoglobin, and a curve L11 represents a molar absorption coefficient of oxygenated hemoglobin. Furthermore, in FIG. 6, a straight line B_{B} represents a wavelength band of the narrow band light emitted by the third light source unit 63, a straight line B_{G} represents a wavelength band of the narrow band light emitted by the second light source unit 62, and a straight line B_{R} represents a wavelength band of the narrow band light emitted by the first light source unit 61.

There are two kinds of oxygenated hemoglobin in hemoglobin in the blood, which are reduced hemoglobin (Hb) not combined with oxygen and hemoglobin (HbO₂) combined with oxygen. The oxygen saturation (SPO₂) used in the first embodiment represents a ratio of the oxygenated hemoglobin in all hemoglobin inside the blood. The oxygen saturation SPO₂ is defined by a following formula (6).

$${SPO}_{2} ( \% ) = \frac{{HbO}_{2}}{{HbO}_{2} + Hb} \times 100 \qquad ( 6 )$$

The oxygen saturation can be calculated by using two wavelengths different from each other by the Beer-Lambert law. In a pulse oximeter used to calculate oxygen saturation in the related art, for example, light of 660 nm and 900 nm are used, and in the case where the two wavelengths which differ from each other are defined as λ1 and λ2, AC components and DC components of signal values respectively obtained are defined as I_{AC}^{λ1}, I_{DC}^{λ1}, I_{AC}^{λ2}, I_{DC}^{λ2}, the oxygen saturation SPO₂ can be expressed by a following formula (7).

$${SPO}_{2} = A \frac{{I}_{AC}^{λ 1} \text{/} {I}_{DC}^{λ 1}}{{I}_{AC}^{λ 2} \text{/} {I}_{DC}^{λ 2}} + B \qquad ( 7 )$$

Here, A and B represent correction coefficients and are preliminarily obtained by performing calibration processing.

In the first embodiment, the oxygen saturation calculation unit 912 calculates the oxygen saturation by acquiring I_{AC}^{λ1}, I_{DC}^{λ1}, I_{AC}^{λ2}, I_{DC}^{λ2} by performing pixel-averaging in a target region. Specifically, in the first embodiment, λ1 is 520 nm (signal G of pixel G), and λ2 is 660 nm (signal R of pixel R). In other words, the oxygen saturation calculation unit 912 calculates the oxygen saturation of the subject by using the signal G of the pixel G (pixel value G) and the signal R of the pixel R (pixel value R) included in the image corresponding to the image data generated by the imaging device 222.

Referring back to FIG. 5, explanation from Step S105 will be continued.

In Step S105, the display controller 913 superimposes the oxygen saturation calculated by the oxygen saturation calculation unit 912 on the color image generated by the color image generation unit 911, and outputs the superimposed image to the display device 4. Consequently, as illustrated in FIG. 7, the display device 4 displays, on a display area 41, a color image P1 on which oxygen saturation W1 is superimposed. As a result, a user can grasp the oxygen saturation of the subject while viewing the color image.

Subsequently, in the case where a command signal to finish observation of the subject is received via the input unit 94 (Step S106: Yes), the endoscope system 1 finishes the processing. In contrast, in the case where the command signal to finish observation of the subject is not received via the input unit 94 (Step S106: No), the endoscope system 1 returns to Step S101.

According to the first embodiment, the light source device 6 emits the narrow band light to the subject, the color image generation unit 911 generates the color image data by using the correction data and the image data generated by the imaging device 222, the oxygen saturation calculation unit 912 calculates the oxygen saturation of the subject by using the pixel value R of the pixel R and the pixel value G of the pixel G included in the image data generated by the imaging device 222, and the display device 4 displays the oxygen saturation superimposed on the color image. Therefore, the color image and the oxygen saturation can be observed simultaneously without upsizing the device.

Furthermore, according to the first embodiment of the present invention, the color image generation unit 911 generates the color image by using the image data generated at the same timing by the imaging device 222, and also the oxygen saturation calculation unit 912 calculates the oxygen saturation. Therefore, the subject can be observed with high accuracy.

### Second Embodiment

Next, a second embodiment of the present invention will be described. An endoscope system according to the second embodiment is different in configurations of a light source device 6 and an image processing device 9 according to the first embodiment, and furthermore, the endoscope system according to the second embodiment updates correction data. In the following, a configuration of the endoscope system according to the second embodiment will be described first, and then the processing executed by the endoscope system according to the second embodiment will be described.

Configuration of Endoscope System

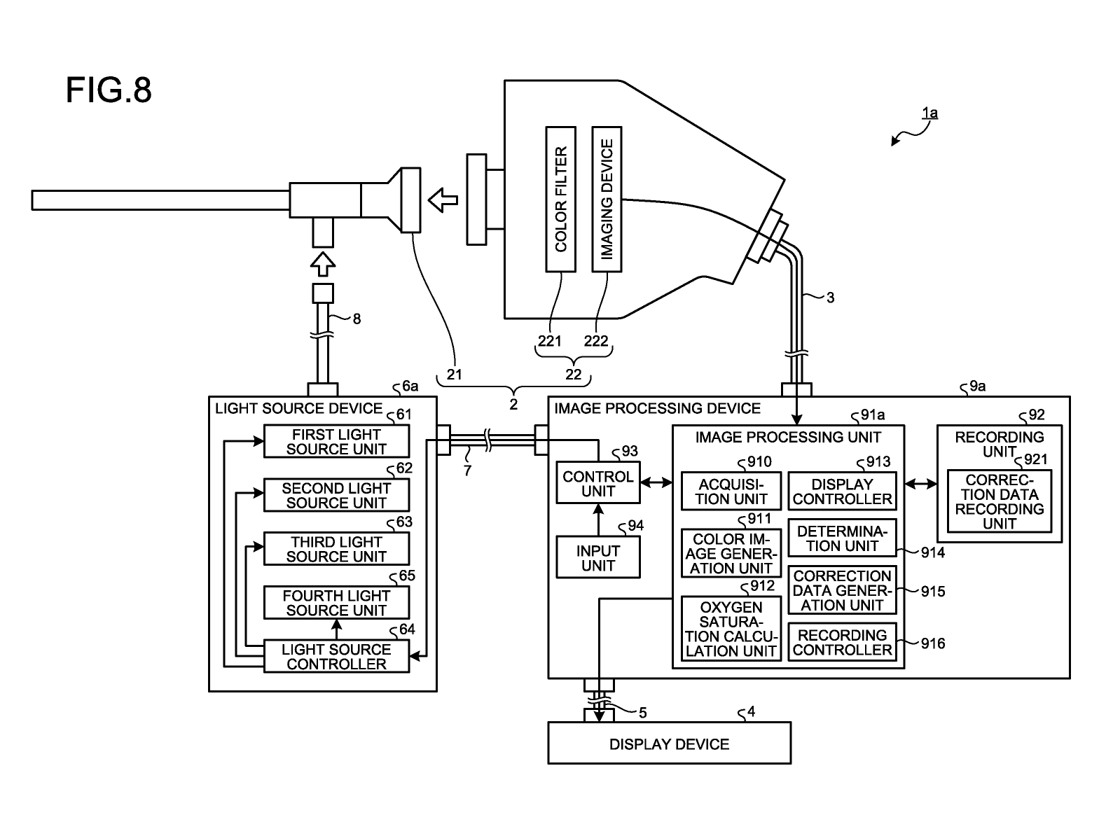

FIG. 8 is a diagram illustrating a brief configuration of the endoscope system according to a second embodiment of the present invention. An endoscope system 1a illustrated in FIG. 8 includes a light source device 6a and an image processing device 9a instead of the light source device 6 and the image processing device 9 of the endoscope system 1 according to the first embodiment.

The light source device 6a includes a fourth light source unit 65 in addition to the configuration of the light source device 6 according to the first embodiment. The fourth light source unit 65 emits white light under the control of a light source controller 64. The fourth light source unit 65 is formed by using a xenon lamp, a white LED lamp, and the like.

An image processing device 9a includes an image processing unit 91a instead of an image processing unit 91 according to the first embodiment. The image processing unit 91a further includes a determination unit 914, a correction data generation unit 915, and a recording controller 916 in addition to a configuration of the image processing unit 91 according to the first embodiment.

The determination unit 914 determines whether the endoscope system 1a is deteriorated based on: second image data generated by the imaging device 222 when white light is emitted; third image data generated by making an imaging device 222 image a calibration chart C1 when white light is emitted to the calibration chart C1 (calibration portion) having a plurality of color patches of a known spectrum; and correction data recorded by a correction data recording unit 921.

The correction data generation unit 915 generates the correction data by using: image data (second image data) generated by the imaging device 222 when the light source device 6a emits white light; and image data (first image data) generated by the imaging device 222 when the light source device 6a emits three kinds of narrow band light.

If the determination unit 914 determines that the endoscope system 1a is deteriorated, the recording controller 916 performs updating by causing the correction data recording unit 921 to record latest correction data generated by the correction data generation unit 915.

Operation of Endoscope System

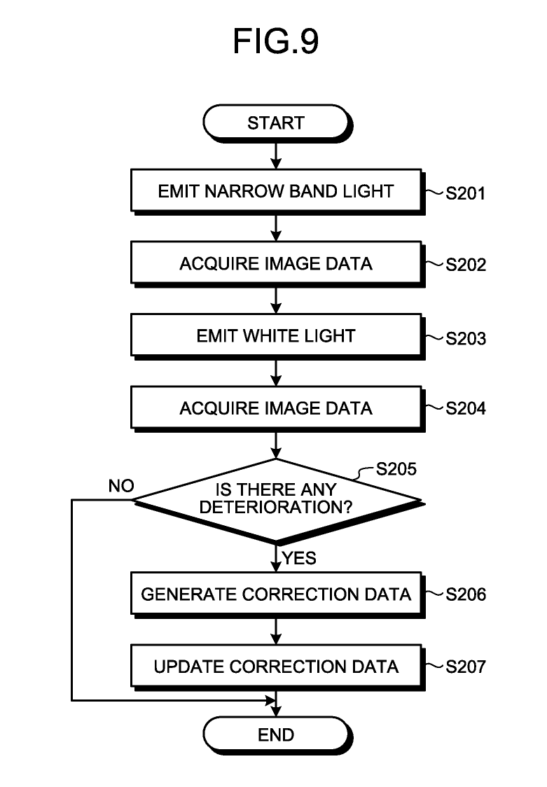

Next, correction data update processing executed by the endoscope system 1a will be described. FIG. 9 is a flowchart illustrating an outline of the correction data update processing executed by the endoscope system 1a. Furthermore, in the case where the endoscope system 1a executes the correction data update processing, the endoscope system 1a emits illumination light to the above-described calibration chart C1 and images the same. Note that the endoscope system 1a according to the second embodiment performs processing same as the endoscope system 1 according to the first embodiment. Specifically, the endoscope system 1a causes the light source device 6a to emit the narrow band light at the time of observing a subject, a color image generation unit 911 generates a color image by using the image data generated by the imaging device 222 and the correction data recorded by the correction data recording unit 921, and a display controller 913 combines the color image with oxygen saturation calculated by an oxygen saturation calculation unit 912 and outputs the same to the display device 4 (refer to FIG. 7).

As illustrated in FIG. 9, a control unit 93 first controls the light source device 6a, thereby making the light source device 6a emit the narrow band light to the calibration chart C1 (Step S201).

Subsequently, an acquisition unit 910 acquires the image data generated by the imaging device 222 when the light source device 6a emits the narrow band light to the calibration chart C1 (Step S202).

After that, the control unit 93 controls the light source device 6a, thereby making the light source device 6a emit white light to the calibration chart C1 (Step S203).

Subsequently, the acquisition unit 910 acquires the image data generated by the imaging device 222 when the light source device 6a emits white light to the calibration chart C1 (Step S204).

After that, the determination unit 914 determines whether the endoscope system 1a is deteriorated (Step S205). Specifically, the determination unit 914 determines whether the light source device 6a and the imaging device 222 are deteriorated based on the image data acquired in Step S202, the image data acquired in Step S204, and the correction data recorded by the correction data recording unit 921. More specifically, the determination unit 914 determines whether an absolute value of a value obtained by subtracting, from image data I2 generated by the imaging device 222 when the light source device 6a emits the white light to the calibration chart C1, a value obtained by multiplying the correction data M by image data I1 generated by the imaging device 222 when the light source device 6a emits the narrow band light to the calibration chart C1 is smaller than a predetermined threshold ε (|I2−I1× M|<ε). In the case where determination unit 914 determines that the endoscope system 1a is deteriorated (Step S205: Yes), the endoscope system 1a proceeds to Step S206. In contrast, in the case where the determination unit 914 determines that the endoscope system 1a is not deteriorated (Step S205: No), the endoscope system 1a finishes the processing.

In Step S206, the correction data generation unit 915 generates the correction data. Specifically, the correction data generation unit 915 generates, as the correction data M, the value obtained by dividing the image data I2 acquired in Step S204 by the image data I1 acquired in Step S202 (I2/I1).

Subsequently, the recording controller 916 performs updating by recording the correction data generated by the correction data generation unit 915 in the correction data recording unit 921 (Step S207). After Step S207, the endoscope system 1a finishes the processing.

According to the second embodiment, the correction data generation unit 915 generates the correction data by using the image data (third image data) when white light is emitted to the calibration chart C1 and the image data (second image data) when the light source device 6a emits the narrow band light. Therefore, a highly-accurate color image and oxygen saturation can be observed simultaneously.

Furthermore, according to the second embodiment, in the case where determination unit 914 determines that the endoscope system 1a is deteriorated, the correction data generation unit 915 generates the correction data. Therefore, the color image generation unit 911 can generate the highly-accurate color image regardless of a deterioration level of the endoscope system 1a.

### Third Embodiment

Next, a third embodiment of the present invention will be described. An endoscope system according to the third embodiment is different in configurations of a camera head 22 and an image processing device 9 according to the first embodiment and also different in processing executed. Specifically, the endoscope system according to the third embodiment displays a fluorescent image in a manner further combined with a color image. In the following, the configuration of the endoscope system according to the third embodiment will be described first, and then the processing executed by the endoscope system according to the third embodiment will be described.

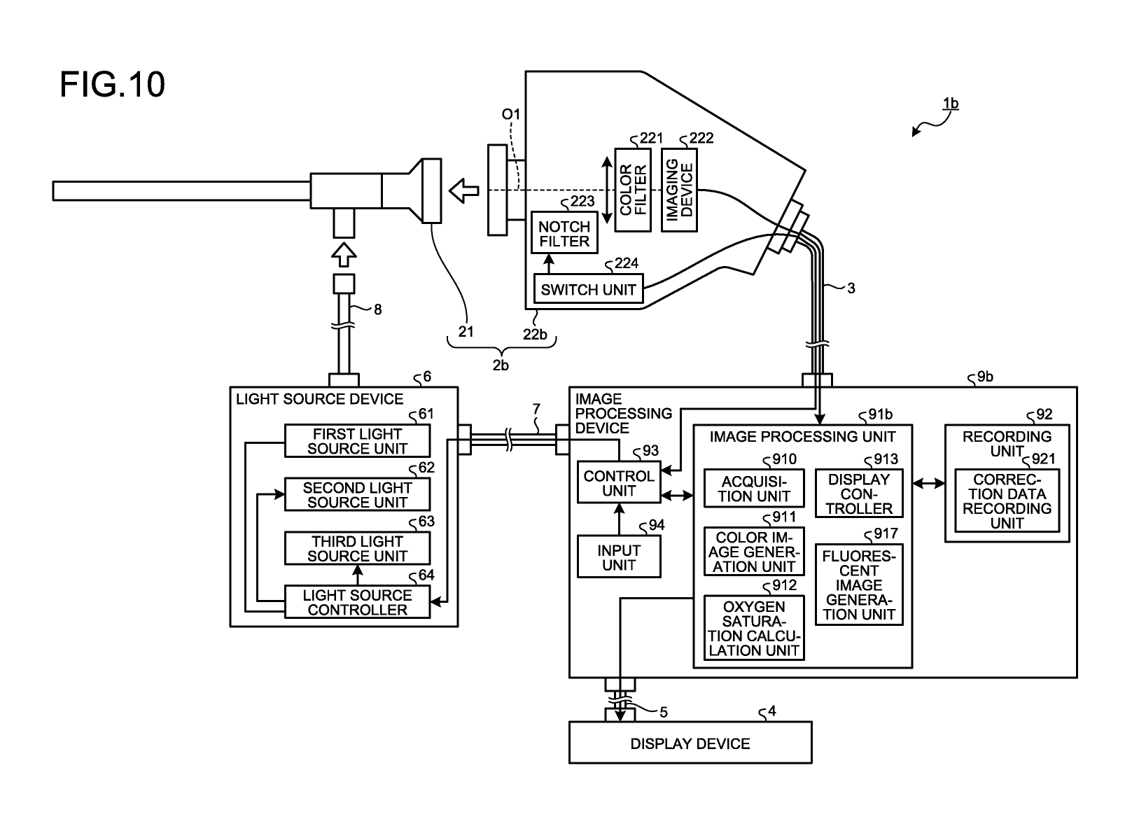

FIG. 10 is a diagram illustrating a brief configuration of the endoscope system according to the third embodiment of the present invention. An endoscope system 1b illustrated in FIG. 10 includes an endoscope 2b and an image processing device 9b instead of an endoscope 2 and the image processing device 9 of an endoscope system 1 according to the first embodiment.

The endoscope 2b includes a camera head 22b instead of the camera head 22 according to the first embodiment.

The camera head 22b includes a notch filter 223 and a switch unit 224 in addition to the configuration of the camera head 22 according to the first embodiment.

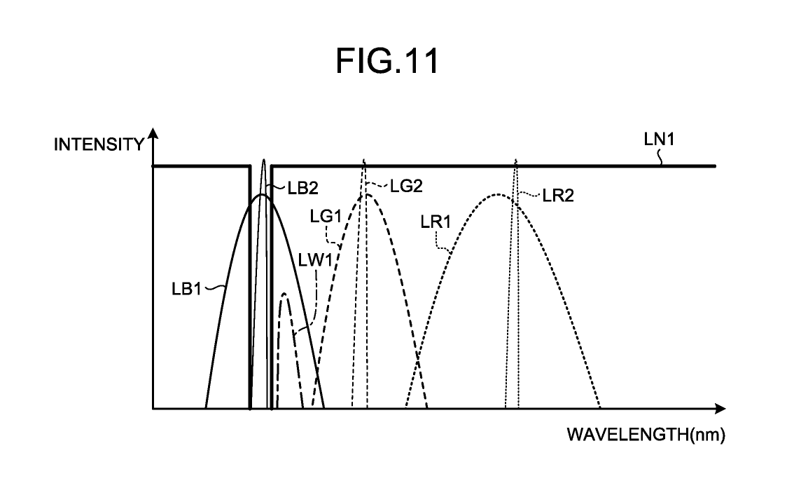

The notch filter 223 passes light of a predetermined wavelength band. FIG. 11 is a diagram illustrating a relation among narrow band light respectively emitted by a first light source unit 61, a second light source unit 62, and a third light source unit 63, respective spectral sensitivity of a pixel B, a pixel G, and a pixel R, and a transmission property of the notch filter 223. Furthermore, in FIG. 11, a curve LB1 represents the spectral sensitivity of the pixel B, a curve LG1 represents the spectral sensitivity of the pixel G, a curve LR1 represents the spectral sensitivity of the pixel R, a curve LB2 represents intensity of the narrow band light emitted by the third light source unit 63, a curve LG2 represents intensity of the narrow band light emitted by the second light source unit 62, and a curve LR2 represents intensity of the narrow band light emitted by the first light source unit 61. Furthermore, in FIG. 11, a curve LW1 represents intensity of fluorescence excited by the narrow band light from the third light source unit 63, and a polygonal line LN1 represents a transmission property of the notch filter 223.

As illustrated in FIG. 11, the notch filter 223 cuts off only the narrow band light emitted by the third light source unit 63 functioning as an excitation light source. Consequently, the pixel B can image only fluorescence excited by the narrow band light emitted by the third light source unit 63. As a medical agent to cause such excitation, there is Lake Placid Blue of T2-MP Evitag, for example. This medical agent has excitation light of 400 nm and fluorescence of 490 nm. The notch filter 223 can change the wavelength band to be cut in accordance with the medical agent causing excitation and the narrow band light.

Referring back to FIG. 10, the explanation for the configuration of the endoscope system 1b will be continued.

The switch unit 224 switches between inserting the notch filter 223 on an optical path of the optical system of an inserting portion 21 and retracting the notch filter 223 from the optical path of the optical system of the inserting portion 21 under the control of the image processing device 9b. The switch unit 224 is formed by using a stepping motor, a DC motor, and the like. The switch unit 224 may be formed by a rotary mechanism adapted to hold the notch filter 223 and insert the same onto an optical path O1 in accordance with rotation.

The image processing device 9b includes an image processing unit 91b instead of an image processing unit 91 according to the first embodiment.

The image processing unit 91b further includes a fluorescent image generation unit 917 in addition to the configuration of the image processing unit 91 according to the first embodiment.

When the light source device 6 emits light of a plurality of narrow bands in the case where the notch filter 223 is inserted onto a light receiving surface of an imaging device 222, the fluorescent image generation unit 917 generates fluorescent image data of a subject based on fourth image data generated by the imaging device 222.

Processing of Endoscope System

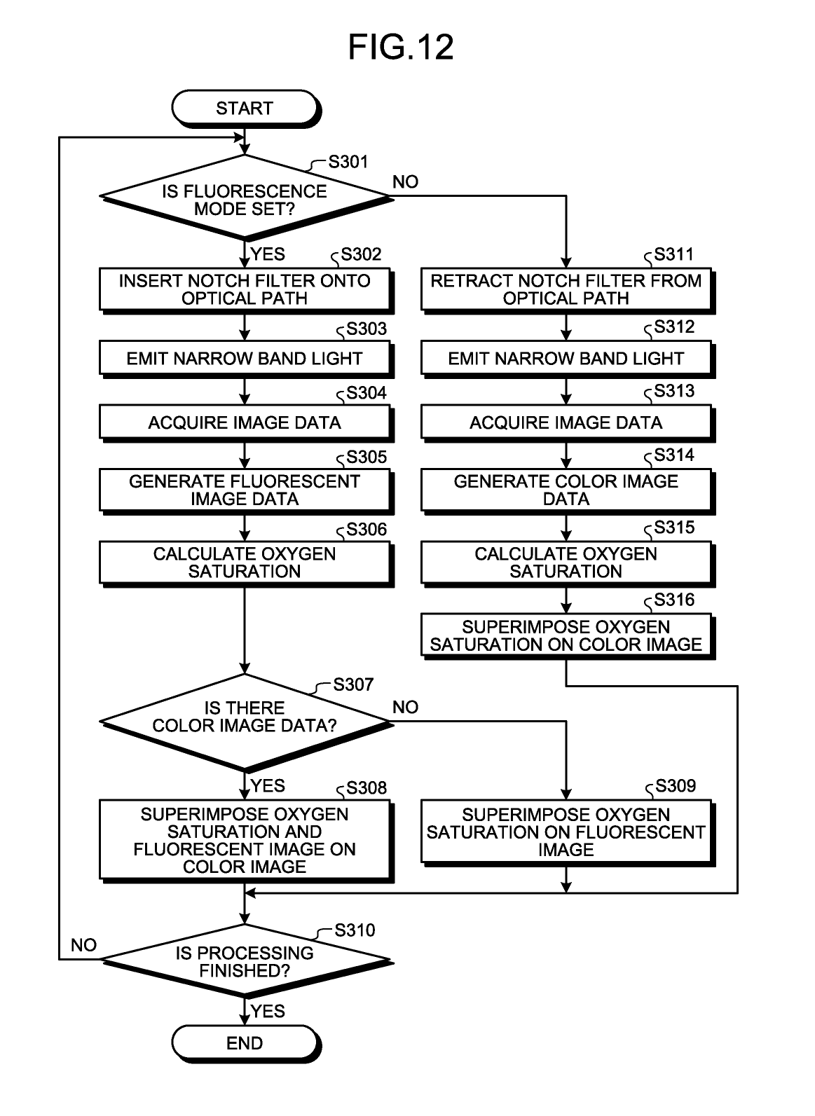

Next, processing executed by the endoscope system 1b will be described. FIG. 12 is a flowchart illustrating an outline of the processing executed by the endoscope system 1b.

As illustrated in FIG. 12, in the case where the endoscope system 1b is set in a fluorescence mode via an input unit 94 (Step S301: Yes), the switch unit 224 first inserts the notch filter 223 onto the optical path O1 of the optical system of the inserting portion 21 under the control of the image processing device 9b (Step S302). After Step S302, the endoscope system 1b proceeds to Step S303 described later.

Steps S303 and S304 correspond to Steps S101 and S102 described above in FIG. 5 respectively.

In Step S305, the fluorescent image generation unit 917 generates fluorescent image data based on a pixel value of the pixel B included in an image corresponding to the fourth image data generated by the imaging device 222. Step S306 corresponds to Step S104 in FIG. 5 described above. After Step S306, the endoscope system 1b proceeds to Step S307.

Subsequently, in the case where there is color image data generated by a color image generation unit 911 in a recording unit 92 immediately before the notch filter 223 is inserted onto the light receiving surface of the imaging device 222, for example, in the case where there is color image data in a previous frame generated by the color image generation unit 911 based on image data generated by the imaging device 222 in a state that the notch filter 223 is not inserted onto the light receiving surface of the imaging device 222 before the fluorescent image generation unit 917 generates the fluorescent image data (Step S307: Yes), the endoscope system 1b proceeds to Step S308 described later. In contrast, in the case where there is no color image data generated by the color image generation unit 911 in the recording unit 92 immediately before the notch filter 223 is inserted onto the light receiving surface of the imaging device 222 (Step S307: No), the endoscope system 1b proceeds to Step S309 described later.

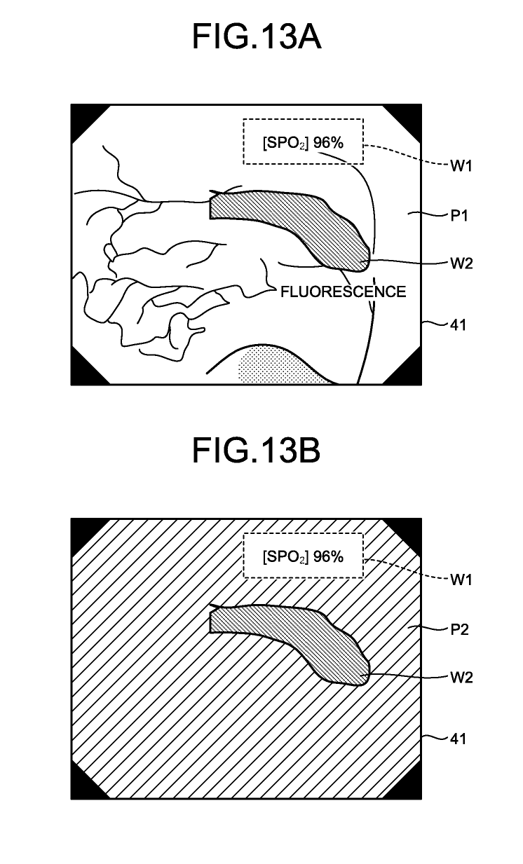

In Step S308, a display controller 913 superimposes oxygen saturation calculated by an oxygen saturation calculation unit 912 and a fluorescent image generated by a fluorescent image generation unit 917 on a color image recorded in the recording unit 92 and generated by the color image generation unit 911, and causes a display device 4 to display the superimposed image. Consequently, the display device 4 can display, as illustrated in FIG. 13A, oxygen saturation W1 and a fluorescent image W2 superimposed on a color image P1. After Step S308, the endoscope system 1b proceeds to Step S310 described later.

In Step S309, the display controller 913 superimposes the oxygen saturation calculated by the oxygen saturation calculation unit 912 on the fluorescent image generated by the fluorescent image generation unit 917, and causes the display device 4 to display the superimposed image. Consequently, the display device 4 can display the oxygen saturation W1 superimposed on the fluorescent image P1 as illustrated in FIG. 13B. After Step S309, the endoscope system 1b proceeds to Step S310 described later.

In Step S310, in the case where a command signal to finish observation of the subject is received from the input unit 94 (Step S310: Yes), the endoscope system 1b finishes the processing. In contrast, in the case where the command signal to finish observation of the subject is not received from the input unit 94 (Step S310: No), the endoscope system 1b returns to Step S301 described.

In Step S301, in the case where the endoscope system 1b is not set in the fluorescence mode via the input unit 94 (Step S301: No), the switch unit 224 retracts the notch filter 223 from the optical path O1 of the optical system of the inserting portion 21 under the control of the image processing device 9b (Step S311).

Steps S312 to S316 correspond to Steps S101 to S105 described above in FIG. 5 respectively. In Step S314, the color image generation unit 911 records, in the recording unit 92, the color image generated by using image data acquired from the camera head 22. After Step S316, the endoscope system 1b proceeds to Step S310.

According to the third embodiment, the fluorescent image, the color image and the oxygen saturation can be observed simultaneously.

### OTHER EMBODIMENTS

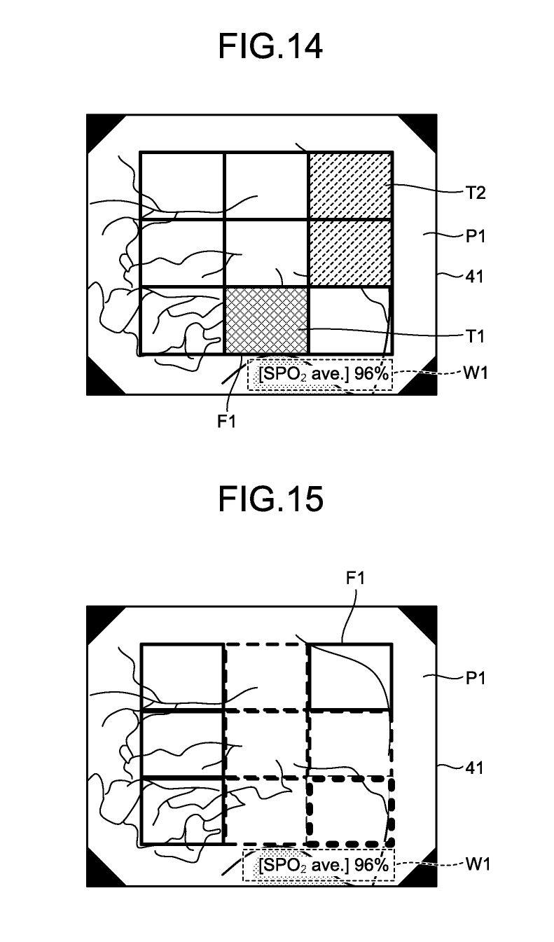

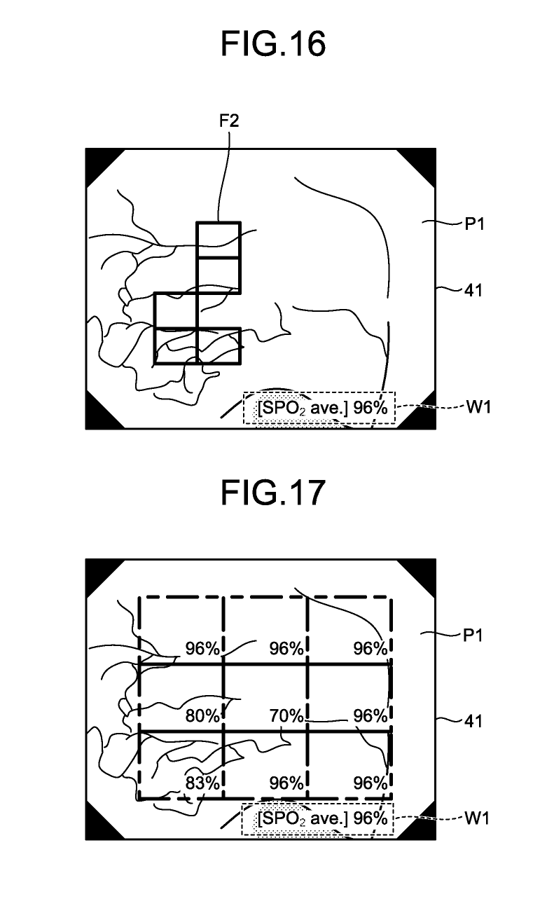

According to first to third embodiments of the present invention, an average value of oxygen saturation in an image corresponding to image data is combined with a color image, but as illustrated in FIG. 14, an oxygen saturation calculation unit 912 may perform division into predetermined regions and calculates oxygen saturation for each of the divided regions, and a display controller 913 may superimpose an average value of a plurality of oxygen saturation calculated by the oxygen saturation calculation unit 912 on a color image. Furthermore, as illustrated in FIG. 14, the display controller 913 may compare the oxygen saturation between the regions and change display modes of a region T1 and a region T2 having oxygen saturation higher than other regions. For example, the display controller 913 may highlight or enhance these regions and cause a display device 4 to display the regions. Moreover, as illustrated in FIG. 15, the display controller 913 may provide a display mode using frames F1 obtained by performing division in accordance with a value of the oxygen saturation, for example, may display the oxygen saturation in ascending order such as red→yellow→green. Also, as illustrated in FIG. 16, the display controller 913 may change a display mode of only a region having a value of the oxygen saturation lower than a threshold, specifically, may provide emphasized display by using a frame F2 (in red, for example). Furthermore, as illustrated in FIG. 17, the display controller 913 may superimpose oxygen saturation calculated by the oxygen saturation calculation unit 912 on a color image P1 for each region, and may cause the display device 4 to display the superimposed image. In this case, the display controller 913 may change a display mode in accordance with the oxygen saturation, for example, may provide display by changing values in ascending order of the oxygen saturation such as red→yellow→green.

In the first to third embodiments, first to third light source units are formed by using light-emitting LEDs, but for example, the light source units may also be formed by using a light source that emits light of a visible light wavelength band and a near-infrared wavelength band like a halogen light source.

Furthermore, in the first to third embodiments, primary color filters of a broad band filter R, a broad band filter G, and a broad band filter B are used, but for example, complementary color filters of magenta, cyan, yellow, and the like may also be used.

In the first to third embodiments, an optical system, a color filter, an imaging device are incorporated in the endoscope, but the optical system, color filter, and imaging device may be housed inside a unit and the unit may be provided detachable to a portable apparatus incorporating an image processing device. Needless to say, the optical system may also be housed inside a lens barrel, and this lens barrel may be formed detachable to a unit housing a color filter, an imaging device, and an image processing unit.

In the first to third embodiments, the oxygen saturation calculation unit is provided in the image processing device, but for example, a function that can calculate oxygen saturation may be implemented by a program or application software in a portable device and a wearable devices such as a watch and a pair of glasses which are capable of performing bidirectional communication, and oxygen saturation of a subject may be calculated in the portable device and the wearable devices by transmitting image data generated by an imaging apparatus.

Moreover, needless to say, the present invention is not limited by the embodiments and various kinds of modification and application can be made within a range of the scope of the present invention. For example, besides the endoscope system used for describing the present invention, the present invention is applicable to any kind of apparatus capable of imaging a subject such as: an imaging apparatus; a portable device and a wearable device including an imaging device in a portable phone or a smartphone; and imaging apparatuses adapted to image a subject through an optical apparatus, such as a video camera, an endoscope, a monitoring camera, and a microscope.

The methods of respective processing by the endoscope systems in the embodiments, namely, all of the processing illustrated in the respective flowcharts may also be stored as a program executable by a control unit such as a CPU. In addition, the program may be distributed by being stored in a storage medium of an external storage device, such as a memory card (ROM card, RAM card, etc.), a magnetic disk, an optical disk (CD-ROM, DVD, etc.), and a semiconductor memory. Then, the control unit such as the CPU reads the program stored in the storage medium of the external storage device, and operation is controlled by the read program, thereby achieving execution of the above-described processing.

The present invention is not limited to the above-described embodiments and modified examples as they are and can be embodied by modifying components within a range without departing from the scope of the invention in the embodying stage. Also, various kinds of inventions can be formed by suitably combining a plurality of components disclosed in the embodiments. For example, some components may be eliminated from all of the components disclosed in the embodiments and modified examples. Furthermore, the components described in the embodiments and modified examples may be suitably combined.

According to some embodiments, the color image and the oxygen saturation can be observed simultaneously without upsizing the device.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
an imaging device having a predetermined array pattern formed by using a pixel R for receiving light of a red wavelength band, a pixel G for receiving light of a green wavelength band, and a pixel B for receiving light of a blue wavelength band, and configured to perform photoelectric conversion on light received by each of the pixel R, the pixel G, and the pixel B to generate image data;
a light source device configured to irradiate a subject with three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively;
a recording unit configured to record correction data for correcting first image data into second image data, the first image data being generated by the imaging device when the light source device irradiates the subject with the three rays of narrow band light, and the second image data being deemed to be generated by the imaging device when white light is emitted;
a color image generation unit configured to generate color image data corresponding to the second image data by using the correction data and the first image data generated by the imaging device when the light source device irradiates the subject with the three rays of narrow band light;
an oxygen saturation calculation unit configured to calculate oxygen saturation of the subject by using a pixel value R of the pixel R and a pixel value G of the pixel G included in the first image data generated by the imaging device when the light source device irradiates the subject with the three rays of narrow band light; and
a display device configured to display a color image corresponding to the color image data generated by the color image generation unit and the oxygen saturation calculated by the oxygen saturation calculation unit.

2. The endoscope system according to claim 1, further comprising a correction data generation unit configured to generate the correction data based on third image data generated by imaging, by the imaging device, a calibration portion having a plurality of patches of a known spectrum when the calibration portion is irradiated with white light, and based on the first image data generated by imaging the calibration portion by the imaging device when the light source device irradiates the calibration portion with the three rays of narrow band light.

3. The endoscope system according to claim 2, further comprising:
a determination unit configured to determine whether at least the light source device is deteriorated, based on the second image data, the third image data, and the correction data recorded by the recording unit; and
a recording controller configured to cause the recording unit to record latest correction data generated by the correction data generation unit to update the correction data if the determination unit determines that the light source device is deteriorated.

4. The endoscope system according to claim 1, further comprising:
a notch filter configured to cut off only one wavelength band of the three rays of narrow band light;
a switch unit configured to switch between inserting the notch filter on a light receiving surface of the imaging device and retracting the notch filter from the light receiving surface of the imaging device; and
a fluorescent image generation unit configured to generate fluorescent image data of the subject based on fourth image data generated by the imaging device when the notch filter is inserted on the light receiving surface of the imaging device and the light source device emits the three rays of narrow band light, wherein
the display device is configured to display the color image data, the oxygen saturation, and the fluorescent image data.

5. The endoscope system according to claim 1, further comprising a display controller configured to superimpose the oxygen saturation calculated by the oxygen saturation calculation unit on a color image corresponding to the color image data generated by the color image generation unit to produce a superimposed image, and configured to cause the display device to display the superimposed image.

6. The endoscope system according to claim 1, wherein the oxygen saturation calculation unit is configured to divide a first image corresponding to the first image data into predetermined regions, and calculate the oxygen saturation for each of the regions.

7. The endoscope system according to claim 1, wherein
the light source device comprises:
a first light source unit configured to emit narrow band light having a wavelength band narrower than that of spectral sensitivity of the pixel R and having a spectrum peak at 660 nm;
a second light source unit configured to emit narrow band light having a wavelength band narrower than that of spectral sensitivity of the pixel G and having a spectrum peak at 520 nm; and
a third light source unit configured to emit narrow band light having a wavelength band narrower than that of spectral sensitivity of the pixel B and having a spectrum peak at 415 nm.

8. An image processing device for performing image processing on image data generated by an imaging device having a predetermined array pattern formed by using a pixel R for receiving light of a red wavelength band, a pixel G for receiving light of a green wavelength band, and a pixel B for receiving light of a blue wavelength band, the image processing device comprising:
an acquisition unit configured to:
acquire correction data for correcting first image data into second image data, the first image data being generated by the imaging device when a subject is irradiated with three rays of narrow band light, the three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively, and the second image data being deemed to be generated by the imaging device when white light is emitted; and
acquire the first image data generated by the imaging device when the subject is irradiated with the three rays of narrow band light;
a color image generation unit configured to generate color image data corresponding to the second image data by using the first image data and the correction data acquired by the acquisition unit; and
an oxygen saturation calculation unit configured to calculate oxygen saturation of the subject by using a pixel value R of the pixel R and a pixel value G of the pixel G included in the image data generated by the imaging device when the subject is irradiated with the three rays of narrow band light.

9. An image processing method for performing image processing on image data generated by an imaging device having a predetermined array pattern formed by using a pixel R for receiving light of a red wavelength band, a pixel G for receiving light of a green wavelength band, and a pixel B for receiving light of a blue wavelength band, the image processing device comprising:
acquiring correction data for correcting first image data into second image data, the first image data being generated by the imaging device when a subject is irradiated with three rays of narrow band light, the three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively, and the second image data being deemed to be generated by the imaging device when white light is emitted;
acquiring the first image data generated by the imaging device when the subject is irradiated with the three rays of narrow band light;
generating color image data corresponding to the second image data by using the first image data and the correction data; and
calculating oxygen saturation of the subject by using a pixel value R of the pixel R and a pixel value G of the pixel G included in the first image data.

10. A non-transitory computer-readable recording medium with an executable program stored thereon for an image processing device, the image processing device being configured to perform image processing on image data generated by an imaging device having a predetermined array pattern formed by using a pixel R for receiving light of a red wavelength band, a pixel G for receiving light of a green wavelength band, and a pixel B for receiving light of a blue wavelength band, the program causing the image processing device to execute:
acquiring correction data for correcting first image data into second image data, the first image data being generated by the imaging device when a subject is irradiated with three rays of narrow band light, the three rays of narrow band light having wavelength bands narrower than those of spectral sensitivity of the pixel R, the pixel G, and the pixel B, respectively, having wavelength bands different from one another, and having spectrum peaks within wavelength bands of the spectral sensitivity of the pixel R, the pixel G, and the pixel B, respectively, and the second image data being deemed to be generated by the imaging device when white light is emitted;
acquiring the first image data generated by the imaging device when the subject is irradiated with the three rays of narrow band light;
generating color image data corresponding to the second image data by using the first image data and the correction data; and
calculating oxygen saturation of the subject by using a pixel value R of the pixel R and a pixel value G of the pixel G included in the first image data.
